(19) United States
(12) Patent Application Publication
(10) Pub. No.: US 2013/0029374 A1
(43) Pub. Date: Jan. 31, 2013

(54) METHOD AND MEASURING POINT FOR MEASURING AT LEAST ONE PHYSICAL AND/OR CHEMICAL, PROCESS VARIABLE OF A MEASURED MEDIUM CONTAINED IN A SINGLE USE CONTAINER

(71) Applicants: Andreas Eberheim, Waldheim (DE); Christian Fanselow, Geringswalde (DE)

(72) Inventors: Andreas Eberheim, Waldheim (DE); Christian Fanselow, Geringswalde (DE)

(73) Assignee: Endress + Hauser Conducta Gesellschaft für Mess- und Regeltechnik mbH + Co. KG, Gerlingen (DE)

(21) Appl. No.: 13/556,405

(22) Filed: Jul. 24, 2012

(30) Foreign Application Priority Data
Jul. 25, 2011 (DE) 10 2011 079 758.0
Aug. 15, 2011 (DE) 10 2011 080 956.2

(51) Int. Cl. G01N 33/483; C12Q 1/06; G01N 21/75; G06F 19/00; G01N 33/50

(52) U.S. Cl. 435/39; 702/19; 436/163; 436/136

(57) ABSTRACT

A method, and a measuring point for performing the method, for measuring at least one physical and/or chemical, process variable of a medium contained in a single use container. A single use sensor is connected with the single use container. The single use sensor with the single use container is sterilized by radiation, especially beta or gamma radiation. An electronics unit is connected with the single use sensor, and the electronics unit is connected with at least one superordinated system via at least one interface. The electronics unit and/or the single use sensor are/is supplied with energy, and measurement data of the single use sensor are registered. The measurement data are processed by the electronics unit and/or the superordinated system, data, including measurement data, are transmitted to the superordinated system and/or received from the superordinated system, and, after termination of one or more measuring cycles, the electronics unit is released from the single use sensor.

The invention relates to a method for measuring at least one physical and/or chemical, process variable of a measured medium contained in a single use container and to a measuring point for performing this method, especially for applications in single use measurements technology for biotech, biochemical, pharmaceutical, biological or food processes.

Pharmaceutical, chemical, biological, biochemical, biotech or food processes are, in increasing measure, performed in single use containers (also referred to as disposables, or disposable bio-reactors). Such single use containers can comprise, for example, flexible containers, e.g. bags, hoses, flexible tubes, or fermenters, respectively bioreactors. Bioreactors, or fermenters, frequently possess supply and drain lines, which can be embodied, for example, as hoses, or flexible tubes. Also rigid sections can be present in the supply and drain lines. After terminating a process, the single use container and/or supply and drain lines can be disposed of. In this way, complex cleaning procedures are avoided. Especially, through the use of single use containers, the risk of cross contaminations is avoided and, therewith, bio- and process safety increased. Single use containers are, as a rule, made of synthetic material, e.g. plastics.

Processes running in single use containers are isolated from the environment. Frequently, germ free conditions are required, and the single use containers are sterilized before introduction of media. Frequently used for this purpose in biochemical, biological, biotechnological and pharmaceutical applications is gamma radiation.

In order to monitor or control the processes, it is necessary to measure physical or chemical, measured variables of the media contained in the container. Variables to be monitored can include, for example, temperature, pH value, cell density, optical transmission or concentration of a chemical substance, for example, a certain kind of ion or a certain element or a certain compound.

In order not to endanger process safety, all objects coming in contact with the medium must be sterilized. This means, for process measurements technology, that the sensors for registering process variables must either measure contactlessly or the parts contacting the medium must be single use parts. The means that all parts contacting the medium must be able to withstand sterilization, for example, by gamma radiation.

In the past, predominantly analog measuring points were used. In such case, sensors are connected via analog lines, most often, cable, with a superordinated system, e.g. a measurement transmitter or a control system. The superordinated system contains, for example, control- and evaluating electronics. In such case, each sensor has its own measurement transmitter. Line resistances and -capacitances weaken and corrupt the measurement signal and mean that the sensor and measurement transmitter cannot be located widely removed from one another.

In more recent times, digital measuring points have become popular. In such case, evaluating- and operating electronics are directly connected with the sensor. In an electronics unit, the measurement signals are digitized and forwarded to a superordinated system as digital data via a digital line, for instance, a digital cable, i.e. a cable for the transport of digital signals. Sensors equipped with electronics units are connected either fixedly or via a plugged connection with the digital cable. An advantage of digital sensors is, furthermore, that a single measurement transmitter can be used for all types of sensors, since the data have already been processed in the sensor, i.e. in the electronics unit of the sensor. Because of digital transmission of the data, moreover, line resistances and -capacitances are less critical than in the case of analog signals, measurement performance is increased and the influence of disturbance variables is minimized.

Digital sensors frequently include, besides the evaluating- and operating electronics, also a memory with data concerning the serial number, date of manufacture, calibration date, etc. of the sensor. Currently, there is still no electronics available that resists gamma radiation. Especially, there is no memory that resists gamma radiation.

An object of the invention is to provide a universal measuring point with at least one digital sensor for single use measurements.

The object is achieved by a method for measuring at least one physical and/or chemical, process variable of a medium contained in a single use container, wherein
a single use sensor is connected releasably or non-releasably with the single use container,
the single use sensor with the single use container is sterilized by radiation, especially beta- or gamma radiation,
an electronics unit is connected releasably directly with the single use sensor,
the electronics unit is connected with at least one superordinated system via at least one interface,
the electronics unit and/or the single use sensor are/is supplied with energy,
measurement data of the single use sensor are registered,
the measurement data are processed by the electronics unit and/or the superordinated system,
data, including measurement data are transmitted to the superordinated system and/or received from the superordinated system, and,
after termination of one or more measuring cycles, the electronics unit is released from the single use sensor.

An option provides that, after performing the last method step, the single use sensor and/or the single use container are disposed of together or separately and, for the next measuring cycle, a new, “clean”, in given cases, sterile, single use sensor and/or a new, “clean”, in given cases, sterile, single use container are applied.

In an advantageous embodiment of the method, the electronics unit and/or the single use sensor is/are supplied with energy via the interface.

The object is furthermore achieved by a measuring point comprising
a single use container for accommodating medium,
a first interface between the single use container and a single use sensor, wherein the interface is so embodied that the single use container and the single use sensor are connected releasably or non-releasably with one another,
a second interface between the single use sensor and an electronics unit, wherein the interface is so embodied that the single use sensor and the electronics unit are connected releasably directly with one another, and
a third interface between the electronics unit and a superordinated system, wherein the interface serves to transmit data, including measurement data, to a superordinated system and/or to receive data, and/or to supply the electronics unit and/or the single use sensor with energy.

Thus, a measuring arrangement is formed, composed of single use container with medium, single use sensor, electronics unit and superordinated system, wherein, in each case, a suitable interface between the individual components of the measuring arrangement is provided.

The object of the invention is fulfilled by the advantages of applying a modular interface concept suitable for digital sensors. It is especially advantageous that single use container and single use sensor can be sterilized while separated from the electronics unit. Through this isolation in the sterilization, the functional abilities of both the single use sensor as well as also the electronics unit are retained, while maintaining the advantages of application of digital sensors.

In a further development, the first interface between the single use container and the single use sensor is so embodied that a first securement unit, especially a coupling nut or a spring clamp, connects the single use container with the single use sensor. By applying a securement unit, an optimal mechanical connection between single use container and single use sensor can be achieved. Moreover, the securement unit is important for the further development of the interface concept, as will be explained in greater detail below.

The single use sensor and the single use container are so embodied that they can be sterilized with radiation, especially gamma- or beta radiation.

In a form of embodiment, the second interface between the single use sensor and the electronics unit is so embodied that a plug contact directly electrically connects the single use sensor with the electronics unit. Because of this direct connection, no supplemental cable or other connection is necessary, whereby space and costs are saved. Additionally, the measuring performance is increased.

In a preferred embodiment, the second interface between the single use sensor and the electronics unit is so embodied that a second securement unit, especially a screw cap, affixes the single use sensor mechanically with the electronics unit. Application of this securement unit provides an optimal mechanical connection between single use sensor and electronics unit. In this case, the second securement unit engages the first securement unit. Thus, are the first and the second securement unit so embodied that an easy, safe and rapid release as well as connecting together are possible.

The single use sensor is, for example, a pH, redox potential, ISFET, temperature, conductivity, pressure, oxygen, especially dissolved oxygen, or carbon dioxide sensor; an ion-selective sensor; an optical sensor, especially a turbidity sensor, a sensor for optical determination of oxygen concentration, or a sensor for determining number of cells and cell structure; a sensor for monitoring certain organic or metal compounds; a sensor for determining concentration of a chemical substance, for example, a certain kind of ion or a certain element or a certain compound; or a biosensor, e.g. a glucose sensor.

In an advantageous embodiment, the electronics unit is composed of at least one subunit, wherein the subunit is an information and/or data memory, an analog to digital converter, a digital to analog converter, a communication unit, a triggering unit, a display, a control unit with inputs and outputs, an alarm system and/or an evaluation unit. Through the application of highly integrated electronics, can all demands can be met on a few, possibly even only one, chip.

In a useful embodiment, the third interface between electronics unit and superordinated system is embodied as a galvanically decoupled interface, especially an inductive, capacitive or optical interface, and the connection to the superordinated system is implemented via an electrical line, especially a cable.

In an alternative embodiment, the third interface between electronics unit and superordinated system is embodied as a wireless interface. In this way, a cable can be omitted and the measuring point can be applied even in difficultly accessible locations. An option is that the measuring point supplementally includes its own energy supply, e.g. a single use battery, a rechargeable battery, a fuel cell or a solar cell. The combination of its own energy supply with a wireless embodiment of the third interface is considered especially advantageous.

In an additional alternative embodiment, the third interface between electronics unit and superordinated system is implemented in the form of fixed cable.

In a preferred embodiment of the measuring point of the invention, the superordinated system is a measurement transmitter, a control system, a PLC or a handheld device.

An option is that electronics unit and superordinated system form a single system. As above explained, a few, possibly a single, electronic chip can, through the application of a highly integrated technology, fulfill a 1 demands.

By digitizing the measurement data relatively early in the measuring arrangement, i.e. directly after the registering thereof, line resistances and -capacitances are less critical than in the case of analog signals, the measuring performance is increased and the influence of disturbances is minimized. An advantage of digital sensors is, furthermore, that one measurement transmitter can be used for different types of sensors, since the data have already been processed in the sensor, or, more specifically, in the electronics unit of the sensor.

The invention will now be explained in greater detail based on the appended drawing, the figures of which show as follows:

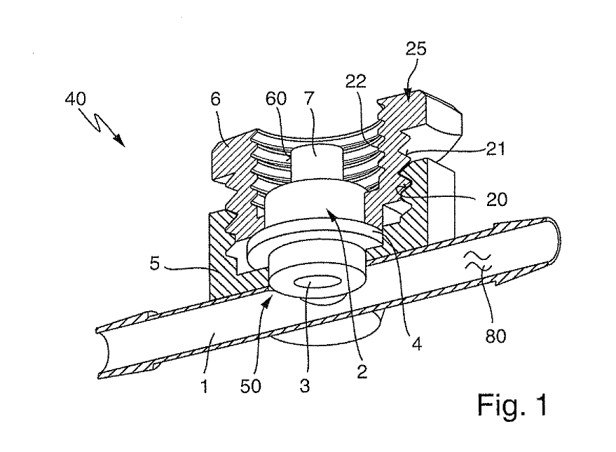

FIG. 1 a cross section through a measuring point with a first interface; and

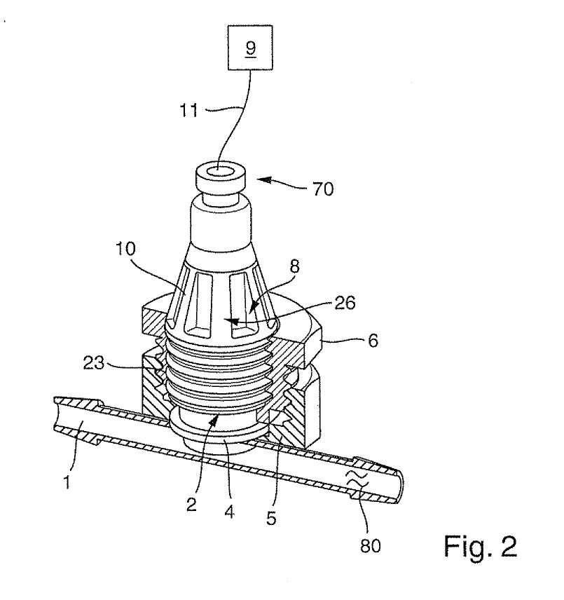

FIG. 2 a cross section through a measuring point with first, second and third interfaces, as well as a superordinated system.

In the figures, equal features are provided with equal reference characters.

FIG. 1 shows a measuring point 40 for registering one or more process variables of a medium 80. In this instance, a single use sensor 2 is connected via a first interface 50 with a single use container 1, through which medium 80 is flowing. The single use container 1 is embodied here as a flowthrough housing. The invention can involve other, even flexible, single use containers, e.g. bag, hoses, or flexible tubes, and fermenters.

The single use sensor 2 can be, for example, a pH sensor or a conductivity sensor. Other options for sensors requiring direct contact with the medium 80 include e.g. redox potential, temperature, pressure, oxygen, especially dissolved oxygen, and carbon dioxide sensors. Furthermore, biosensors, for example, glucose sensors and the like or sensors for determining a concentration of a chemical substance, for example, a certain kind of ion or a certain element or a certain compound, can also be used. Other options include sensors, which do not require direct contact with the medium 80, e.g. optical sensors, for example, turbidity sensors, sensors for optical determining oxygen concentration, sensors for monitoring certain organic or metal compounds or sensors for determining the number of cells and cell structures.

Single use sensor 2 contacts medium 80 via the process contacting part 3. Via a seal 4, it is assured that no medium from the single use container 1 can flow past the single use sensor 2 and that no impurities can penetrate in the reverse direction into the single use container 1. For seating the single use sensor 2, an adapter 5 is located on the single use container 1. Adapter 5 is an integral part of the single use container 1 and has a cavity for the seal 4, as well as an internal thread 20. An option is to weld or otherwise secure the measuring point to a nozzle or similar access port to the medium to be measured. In this case, the adapter 5 includes corresponding securement means corresponding to the weldable nozzle or the like.

After mounting the single use sensor 2, a coupling nut 6 with an external thread 21 matching the internal thread 20 of the adapter 5 assures the mechanical mounting of single use sensor 2 to single use container 1. Additionally, coupling nut 6 has an internal thread 22 for the subsequent mounting of an electronics unit 8. Alternatively, instead of a solution with matching threads 20, 21, a spring operated clamp can be used for securing the single use sensor 2 to the single use container 1.

Located on the side of the single use sensor 2 facing away from the medium 80 is a plug contact 7 on the single use sensor 2 for later electrical contacting of the electronics unit 8.

The components are sterilized in this state (i.e. single use sensor 2 secured to the single use container 1 by the coupling nut 6). The sterilization is done typically with beta or gamma radiation.

Materials for the parts to be sterilized include, especially, synthetic materials, or plastics, for example, polypropylene (PP). The use of synthetic material makes components, on the one hand, almost unbreakable. On the other hand, such a housing of synthetic material can be produced with established injection molding methods at favorable costs, a feature which is especially advantageous in the case of single use parts. Especially the application of PP enables sterilization by means of beta and gamma radiation.

For a second interface 60, the electronics unit 8 is connected in FIG. 2 with the single use sensor 2 by means of a screw cap 10. The electronics unit 8 has a receptacle corresponding to the plug contact 7 of the single use sensor 2, in order to provide electrical contact between the electronics unit 8 and the single use sensor 2. The screw cap 10 has an external thread 23 corresponding to the internal thread 22 of the coupling nut 6, in order to effect the mechanical connection. An option is that the electronics unit 8 is integrated into the screw cap 10.

In such case, the first and the second interfaces 50, 60 are from the mechanical point of view so embodied that easy, safe and rapid disengagement as well as securement are possible.

The outer material of the electronics unit 8 is preferably a synthetic material. Another option is a metal housing.

Electronics unit 8 includes, for example, an information- and/or data memory, an analog to digital converter, a digital to analog converter, a communication unit, a triggering unit, a display, a control unit with input and output, an alarm system and/or an evaluation unit.

A third interface 70 connects the electronics unit 8 with a superordinated system 9 through a digital cable 11. Instead of a cable 11, also wireless transmission means, e.g. wireless HART or WLAN, can be used.

A superordinated system 9 in the sense of this invention comprises a PLC, a measurement transmitter, a control system, a handheld device or, in the simplest case, a display for display of measurement data.

An option is to integrate the electronics unit 8 in the superordinated system 9 (or also, conversely, the superordinated system 9 into the electronics unit 8). Through the application of a highly integrated technology, many or all of the electronic tasks (e.g. analog to digital-conversion, etc.) can be cared for directly in the electronics unit 8

The third interface 70 is embodied as a galvanically decoupled interface, especially an inductive interface. Other possible options are a capacitive or optical interface. Alternatively, a fixed cable can be used, in the case of which the electronics unit is integrated into the one side of the fixed cable and the other side has a suitable interface for connection to a superordinated system. Via the third interface 70 are transmitted data bidirectionally and energy unidirectionally. An option is that the measuring point is equipped with its own energy supply, e.g. by way of a single use battery, a rechargeable battery, a fuel cell or a solar cell.

After assembly of the components, single use container 1, single use sensor 2 and coupling nut 6, these are sterilized. Then, the electronics unit 8 is connected with the single use sensor 2 via the plug contact 7 and secured via the screw cap 10. Next, the pharmaceutical, chemical, biological, biochemical, biotech or food process to be monitored is started. In such case, the single use sensor 2 measures via the process contacting part 3 one or more process variables of the medium 80 in the single use container 1. The electronics unit 8 processes these measurement data, including, among others, digitizing the measurement data and forwarding such via an interface to the superordinated system 9.

After termination of a process and, thus, a measurement, the electronics unit 8 is removed from the single use sensor 2 through releasing the coupling nut 6. The originally sterilized parts, including single use container 1, single use sensor 2 and coupling nut 6, can now be disposed of following termination of the process. An option is to reuse the coupling nut 6, since it does not contact medium.

For an additional process, and, thus, measuring cycle, newly sterilized components (i.e. single use container 1, single use sensor 2 and, in given cases, coupling nut 6) are used. Since the electronics unit 8 was never in contact with the medium 80, it, as well as also all other components not contacting the medium, can be used again.

### LIST OF REFERENCE CHARACTERS

1 single use container
2 single use sensor
3 process contacting part of 2
4 seal
5 adapter
6 coupling nut
7 plug contact
8 electronics unit
9 superordinated system
10 screw cap
11 digital cable
20 internal thread of 5
21 external thread of 6
22 internal thread of 6
23 external thread of 10
25 securement unit
26 securement unit
40 measuring point
50 interface between 1 and 2
60 interface between 2 and 8
70 interface between 8 and 9
80 medium

1-13. (canceled)

14. A method for measuring at least one physical and/or chemical, process variable of a medium contained in a single use container, comprising the steps of:
connecting a single use sensor releasably or non-releasably with the single use container;
sterilizing the single use sensor with the single use container by radiation, especially beta or gamma radiation;
connecting an electronics unit releasably directly with the single use sensor;
connecting the electronics unit with at least one superordinated system via at least one interface;
supplying the electronics unit and/or the single use sensor with energy;
registering the measurement data of the single use sensor;
processing the measurement data by the electronics unit and/or the superordinated system;
transmitting data, including measurement data, to the superordinated system and/or received from the superordinated system; and,
releasing the electronics unit from the single use sensor after termination of one or more measuring cycles.

15. The method as claimed in claim 14, wherein:
the electronics unit and/or the single use sensor is/are supplied with energy via the interface.

16. A measuring point for performing a method for:
measuring at least one physical and/or chemical, process variable of a medium contained in a single use container, comprising the steps of: connecting a single use sensor releasably or non-releasably with the single use container; sterilizing the single use sensor with the single use container by radiation, especially beta or gamma radiation; connecting an electronics unit releasably directly with the single use sensor; connecting the electronics unit with at least one superordinated system via at least one interface; supplying the electronics unit and/or the single use sensor with energy; registering the measurement data of the single use sensor; processing the measurement data by the electronics unit and/or the superordinated system; transmitting data, including measurement data, to the superordinated system and/or received from the superordinated system; and, releasing the electronics unit from the single use sensor after termination of one or more measuring cycles, the measuring point comprising:
a single use container for accommodating medium;
a first interface between said single use container and a single use sensor, wherein said first interface is so embodied that said single use container and said single use sensor are connected releasably or non-releasably with one another;
a second interface between said single use sensor and an electronics unit, wherein said second interface is so embodied that said single use sensor and said electronics unit are connected releasably directly with one another; and
a third interface between said electronics unit and a superordinated system, wherein said third interface serves to transmit data, including measurement data, to said superordinated system and/or to receive data, and/or to supply said electronics unit and/or said single use sensor with energy.

17. The measuring point as claimed in claim 16, wherein:
said first interface between said single use container and said single use sensor is so embodied that a first securement unit, especially a coupling nut or a spring clamp, connects said single use container with said single use sensor.

18. The measuring point as claimed in claim 16, wherein:
said single use sensor and said single use container are so embodied that they can be sterilized with radiation, especially gamma- or beta radiation.

19. The measuring point as claimed in claim 16, wherein:
said second interface between said single use sensor and said electronics unit is so embodied that a plug contact directly electrically connects said single use sensor with said electronics unit.

20. The measuring point as claimed in claim 16, wherein:
said second interface between said single use sensor and said electronics unit is so embodied that a second securement unit, especially a screw cap, affixes said single use sensor mechanically with said electronics unit.

21. The measuring point as claimed in claim 16, wherein:
said single use sensor is a pH, redox potential, ISFET, temperature, conductivity, pressure, oxygen, especially dissolved oxygen, or carbon dioxide sensor; an ion-selective sensor; an optical sensor, especially a turbidity sensor, a sensor for optical determination of oxygen concentration, or a sensor for determining number of cells and cell structures; a sensor for monitoring certain organic or metal compounds; a sensor for determining concentration of a chemical substance, for example, a certain kind of ion or a certain element or a certain compound; or a biosensor, especially a glucose sensor.

22. The measuring point as claimed in claim 16, wherein:
said electronics unit is composed of at least one subunit, said subunit is an information- and/or data memory, an analog to digital converter, a digital to analog converter, a communication unit, a triggering unit, a display, a control unit with inputs and outputs, an alarm system and/or an evaluation unit.

23. The measuring point as claimed in claim 16, wherein:
said third interface between said electronics unit and said superordinated system is embodied as a galvanically decoupled interface, especially as an inductive, capacitive or optical interface, and the connection to the superordinated system is implemented via an electrical line, especially a cable.

24. The measuring point as claimed in claim 16, wherein:
said third interface between said electronics unit and said superordinated system is embodied as a wireless interface.

25. The measuring point as claimed in claim 16, wherein:
said third interface between said electronics unit and said superordinated system is embodied as a fixed cable.

26. The measuring point as claimed in claim 16, wherein:
said superordinated system is a measurement transmitter, a control system, a PLC or a handheld device.
